(19) United States
(12) Patent Application Publication
(10) Pub. No.: US 2019/0317068 A1
(43) Pub. Date: Oct. 17, 2019

(54) Device for the Detection of Conditions Indicative of Illicit Drug Production

(71) Applicant: P Alert Limited, Hamilton (NZ)

(72) Inventors: Robert David Beck, Hamilton (NZ); James Hanson, Hamilton (NZ)

(21) Appl. No.: 16/472,589

(22) PCT Filed: Dec. 21, 2017
(86) PCT No.: PCT/NZ2017/050170

§ 371 Date: Jun. 21, 2019

(30) Foreign Application Priority Data
Dec. 22, 2016 (NZ) 727911

(51) Int. Cl. G01N 33/00; H04Q 9/00

(52) U.S. Cl. CPC G01N 33/0047; H04Q 2209/823; H04Q 9/00; G01N 33/0063

(57) ABSTRACT

A device for the detection of conditions indicative of illicit drug production. The device includes an airborne ethanol sensor circuit configured to output a signal indicative of airborne ethanol concentration. A processor is coupled to the airborne ethanol sensor circuit, configured to receive the signal indicative of airborne ethanol concentration. The processor is configured to determine whether a value of the signal exceeds a predetermined threshold. A communication circuit is coupled to the processor, configured to send a message to at least one remote address on determination of the value of the signal exceeding the predetermined threshold.

### STATEMENT OF CORRESPONDING APPLICATIONS

This application is based the provisional specification filed in relation to New Zealand Patent Application No. 727847, the entire contents of which are incorporated herein by reference.

### TECHNICAL FIELD

The present disclosure relates to a device for the detection of conditions indicative of illicit drug production, more particularly the use of an ethanol sensor to detect conditions indicative of the production of methamphetamine.

### BACKGROUND

The production of illicit drugs such as methamphetamine (also commonly known as “meth” or “P”) is increasingly being performed in rental properties. This results in these properties becoming contaminated by the organic solvents, acids, alkalis and other chemicals used in, and produced by, the production process. This presents a significant health risk to subsequent residents of such contaminated properties.

Further, there is a significant economic cost to the owners of such properties in the testing for contamination, and subsequent decontamination if required. In addition to the actual costs of carrying out these processes, there is also an opportunity cost in terms of lost rental income during this time—along with the stigma of a property having previously been used as a drug lab to the point of needing decontamination.

Sensing units for the detection of indicators of methamphetamine production are known—however have practical limitations which may reduce their effectiveness. For example, one such device is advertised as being a visible and therefore overt deterrent and having anti-tamper sensors to prevent interference. However, it is likely that such a device may be circumnavigated by a motivated party without interfering with the device itself, for example by sealing off the immediate vicinity of the device.

It is an object of the present invention to address the foregoing problems or at least to provide the public with a useful choice.

Further aspects and advantages of the present invention will become apparent from the ensuing description which is given by way of example only.

### SUMMARY OF THE DISCLOSURE

According to an exemplary embodiment of the present disclosure there is provided a device for the detection of conditions indicative of illicit drug production, including:

an airborne ethanol sensor circuit configured to output a signal indicative of airborne ethanol concentration;

a processor coupled to the airborne ethanol sensor circuit, configured to:
receive the signal indicative of airborne ethanol concentration;
determine whether a value of the signal exceeds a predetermined threshold;

a communication circuit coupled to the processor, configured to send a message to at least one remote address on determination of the value of the signal exceeding the predetermined threshold.

According to an exemplary embodiment of the present disclosure there is provided a method for the detection of conditions indicative of illicit drug production, including:

driving an airborne ethanol sensor circuit to output a signal indicative of airborne ethanol concentration;

receiving the signal indicative of airborne ethanol concentration at a processor;

determining whether a value of the signal exceeds a predetermined threshold; and

sending a message to at least one remote address on determination of the value of the signal exceeding the predetermined threshold.

In an exemplary embodiment, the device may be powered by a self-contained power source, for example a battery. This may enable the device to be positioned at any desired location within the property without reliance on connection to mains power supply. It may be desirable to reduce power consumption of the device, and therefore longevity of the battery without replacement or recharging, in order to enable a battery of a smaller capacity (and therefore smaller physical footprint) to be used.

Ethanol is used in significant quantities in the manufacture of many illicit drugs, including methamphetamine, and as such it is envisaged that detection of may be used as a reliable indicator of the early stages of a clandestine drug laboratory operating in the premises in which the device is installed—or at least activity within the premises requiring investigation by the property owner or manager. In an exemplary embodiment, the airborne ethanol sensor circuit may include a gas ethanol sensor.

In an exemplary embodiment, the ethanol sensor may be a metal oxide type gas sensor. By way of example, the gas ethanol sensor may be a tin oxide (SnO₂) sensor such as the MQ3 gas ethanol sensor manufactured by a variety of entities. It is envisaged that such metal oxide based sensors may achieve a desired degree of accuracy within the ethanol concentration levels required, while also having a compact package and relatively low power consumption.

In an exemplary embodiment, the airborne ethanol sensor circuit may be coupled to a sensor driving circuit. In an exemplary embodiment, the sensor driving circuit may be configured to deliver a pulse power up voltage to the gas ethanol sensor to heat the sensor prior to a reading of the sensor being taken. Such a pulse power up voltage delivers pulses of higher current to the resistive element of the sensor, with the higher current heating the resistive element while the pulsing maintains a similar overall power consumption rate in comparison with heating the element with a continuous voltage at a lower current. Many gas ethanol sensors, particularly metal oxide type sensors, require preheating of the sensor before a stable measurement can be achieved. By providing a pulse power up voltage, it has been identified that the preheating time may be reduced (from a recommended time of 24 hours for the MQ3 sensor to about 1 minute) with a tolerable degradation of measurement accuracy (in the order of +/−15%). In doing so, readings may be obtained at large intervals with reduced power consumption.

In an exemplary embodiment, the airborne ethanol sensor circuit may provide a high ohmic burden on the output of the gas ethanol sensor. It is envisaged that this may result in a lower precision in the readings (essentially by reducing the voltage range from which ADC readings are obtained), but lower current draw may assist with reducing power consumption.

In an exemplary embodiment, the processor may be programmed to take periodic readings at any desired time interval, for example from every few minutes, to days, or weeks. In exemplary embodiments, the processor may be configured to control the sensor driving circuit to preheat the sensor prior to taking a reading—whether using the pulse power up as described above, or a constant voltage power up over a longer period of time.

In an exemplary embodiment, the predetermined threshold for comparison with the value of the signal may be indicative of the airborne ethanol (C₂H₅OH) concentration being above about 75 ppm. It is envisaged that this threshold may avoid triggering of an alert in common household activity such as cleaning using rubbing alcohol or drinking alcohol consumption. It should be appreciated that this value is not intended to be limiting to all exemplary embodiments, as it is envisaged that in an exemplary embodiment the predetermined threshold may be above about 60 ppm. In an exemplary embodiment, the predetermined threshold may be above about 100 ppm.

In an exemplary embodiment, the communication circuit may include a wireless modem configured to communicate over a cellular network. For example, the wireless modem may be a GSM modem configured to operate using a SIM card to access a cellular network—although it should be appreciated that the term “GSM” is used by way of illustration and is not intended to limit embodiments of the present disclosure to the GSM standard.

In an exemplary embodiment, the message sent by the communication circuit may uniquely identify the device from which the message was sent.

In an exemplary embodiment, the processor may be configured to disable the modem while an alarm condition is not detected. In doing so, power consumption of the device may be reduced in order to preserve battery life.

In an exemplary embodiment, the processor may be configured to enable the modem and send status messages at predetermined instances when an alarm condition is not detected. For example, the processor may be configured to issue a status message at predetermined dates or times in order to confirm that it is operational.

In an exemplary embodiment, the processor may be configured to enable the modem and listen for command messages at predetermined instances. The command messages may, for example, be used to adjust the predetermined threshold. For example, the processor may be configured to listen for command messages for a predetermined period of time following sending an alert message. In another exemplary embodiment, the processor may be configured to enable the modem and listen for commands at a predetermined date/and or time.

In an exemplary embodiment, a central controller may be provided for communicating with the device. For example, the central controller may be configured to issue command messages to individual devices in order to adjust configuration settings such as the predetermined threshold. This may be, for example, in response to receiving a predetermined number of alerts subsequently designated as being false negatives.

For a firmware and/or software (also known as a computer program) implementation, the techniques of the present disclosure may be implemented as instructions (for example, procedures, functions, and so on) that perform the functions described. It should be appreciated that the present disclosure is not described with reference to any particular programming languages, and that a variety of programming languages could be used to implement the present invention. The firmware and/or software codes may be stored in a memory, or embodied in any other processor readable medium, and executed by a processor or processors. The memory may be implemented within the processor or external to the processor.

A general purpose processor may be a microprocessor, but in the alternative, the processor may be any suitable processor, controller, microcontroller, or state machine. A processor may also be implemented as a combination of computing devices, for example, a combination of a digital signal processor (DSP) and a microprocessor, a plurality of microprocessors, one or more microprocessors in conjunction with a DSP core, or any other such configuration. The processors may function in conjunction with servers and network connections as known in the art.

The steps of a method, process, or algorithm described in connection with the present disclosure may be embodied directly in hardware, in a software module executed by one or more processors, or in a combination of the two. The various steps or acts in a method or process may be performed in the order shown, or may be performed in another order. Additionally, one or more process or method steps may be omitted or one or more process or method steps may be added to the methods and processes. An additional step, block, or action may be added in the beginning, end, or intervening existing elements of the methods and processes.

The above and other features will become apparent from the following description and the attached drawings.

### BRIEF DESCRIPTION OF THE DRAWINGS

The detailed description of the drawings refers to the accompanying figures in which:

FIG. 1 illustrates an exemplary system in accordance with one aspect of the present disclosure;

FIG. 2 is a schematic diagram of an exemplary detector device in accordance with one aspect of the present disclosure; and

FIG. 3 is a flow diagram illustrating an exemplary method of operating the detection device;

FIG. 4 is a flow diagram illustrating a sub-process in the method of operating the detection device, and

FIG. 5 is a flow diagram illustrating a method of configuring the detection device.

### DETAILED DESCRIPTION OF THE DRAWINGS

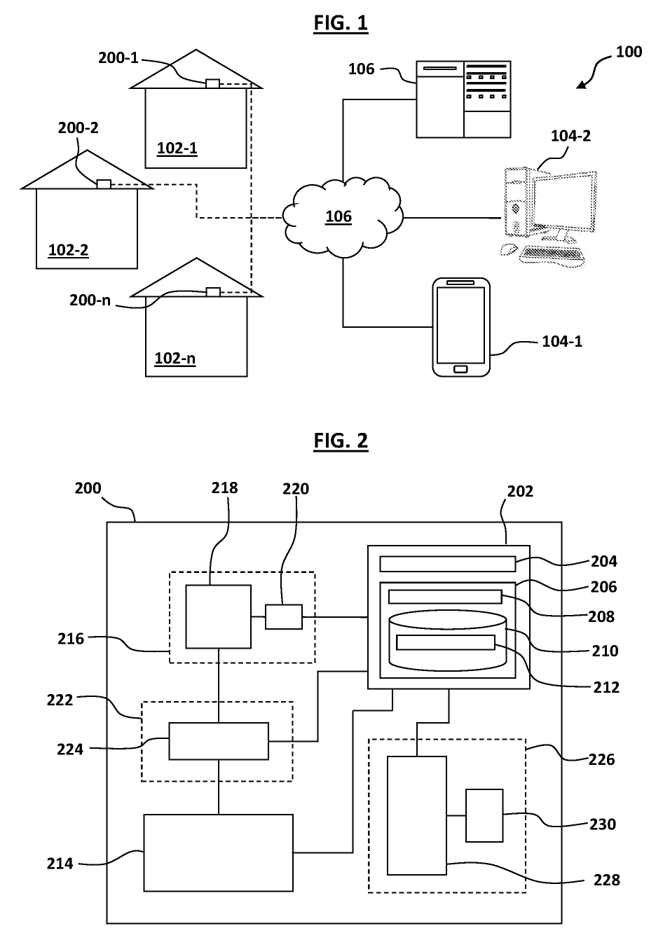

FIG. 1 illustrates an exemplary embodiment of a system 100 within which the present disclosure may operate. Within the system, detector devices 200-1 to 200-n configured to detect conditions indicative of illicit drug production are installed in buildings 102-1 to 102-n (whether private residences or commercial buildings), and communicate with user devices 104 (for example mobile phone 104-1, or computer 104-2) over a cellular network 106. It should be appreciated that reference to cellular network 106 is not intended to limit communication to cellular communication enabled devices, but that communications may be delivered over a variety of networks interfacing with the cellular network 106.

Those communications may include status messages from the detector devices 200-1 to 200-n—including alert messages regarding the detection of conditions indicative of illicit drug production, and operational messages regarding the current operational status of the device 200-1 to 200-n—and command messages to reconfigure the detector devices 200-1 to 200-n.

In an exemplary embodiment, the system 100 may include a command center device 108, for example a server device, which may be used to monitor communications to and from the devices 200-1 to 200-n and potentially issue command messages to the devices 200-1 to 200-n rather than these issuing directly from the user devices 104. For example, the command center device 108 may determine that alert messages issued by one of the devices 200-1 to 200-n are false negatives (whether on analysis of the messages or on instruction of a user), and issue a command message to that device to adjust the settings of that device 200-1 to 200-n. It is also envisaged that in exemplary embodiments the command center device may act as an intermediary between the devices 200-1 to 200-n and the user devices 104, rather than allowing direct communication.

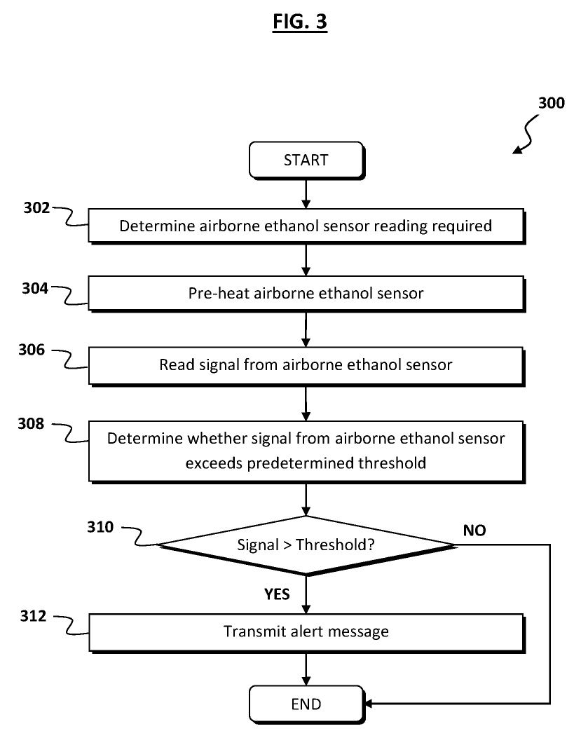

Referring to FIG. 2, the device 200 includes a controller 202. In the exemplary embodiment illustrated, the controller 202 has a processor 204, memory 206, and other components typically present in such computing devices. In the exemplary embodiment illustrated the memory 206 stores information accessible by processor 204, the information including instructions 208 that may be executed by the processor 204 and data 210 that may be retrieved, manipulated or stored by the processor 204. The memory 206 may be of any suitable means known in the art, capable of storing information in a manner accessible by the processor 204, including a computer-readable medium, or other medium that stores data that may be read with the aid of an electronic device.

The processor 204 may be any suitable device known to a person skilled in the art. In an exemplary embodiment, the controller 202 may be a microcontroller, for example the STM8L151F3 microcontroller manufactured by STMicroelectronics. Although the processor 204 and memory 206 are illustrated as being within a single unit, it should be appreciated that this is not intended to be limiting, and that the functionality of each as herein described may be performed by multiple processors and memories, that may or may not be remote from each other.

The instructions 208 may include any set of instructions suitable for execution by the processor 204. For example, the instructions 208 may be stored as computer code on the computer-readable medium. The instructions 208 may be stored in any suitable computer language or format.

Data 206 may be retrieved, stored or modified by processor 204 in accordance with the instructions 208. The data 210 may also be formatted in any suitable computer readable format. Again, while the data 210 is illustrated as being contained at a single location, it should be appreciated that this is not intended to be limiting—the data 210 may be stored in multiple memories or locations.

The data 210 stored on server may include a record of control routines 212 for the device 200. For example, control routines 212 may be provided for driving an ethanol sensor, reading sensor, and managing communication.

The device 200 includes a self-contained power source, for example battery 214. The device 200 also has an airborne ethanol sensor circuit 216 including an ethanol sensor 218 configured to output a signal indicative of airborne ethanol concentration. In an exemplary embodiment, the ethanol sensor 218 may be a metal oxide ethanol sensor. For example, the metal oxide ethanol sensor may be a tin oxide ethanol sensor—an example of which is the MQ3 gas ethanol sensor (manufactured by Hanwei Electronics Co. Ltd). The airborne ethanol sensor circuit 216 also includes a high ohmic load 220 (for example a 10 kΩ resistor) on the output of the sensor 218 to the controller 202.

The ethanol sensor 218 is driven by a sensor driving circuit 222 including a Low Dropout (LDO) linear regulator 224 (for example the MCP1726 manufactured by Microchip Technology Inc.). The regulator 224 is controlled by the controller 202 to deliver a pulse power up voltage to the ethanol sensor 218 to heat the sensor 218 prior to a reading of the sensor 218 being taken by the controller 202.

The device 200 also includes a communication circuit 226 including a wireless modem 228 configured to communicate over a cellular network 106, and coupled to a SIM card 230.

Referring to FIG. 3, a method 300 of operating the device 200 is illustrated. In a first step 302, the controller 202 determines that the sensor 218 is to be read—for example on a predetermined period of time lapsing following a previous reading. In a second step 304 the processor controls the sensor driving circuit 222 to preheat the sensor 218 prior to taking a reading.

In a third step 306 the controller 202 reads the output value of the sensor 218 and converts it to an ADC value indicative of the current airborne ethanol concentration at the sensor 218. In fourth step 308 the controller 202 compares the sensor ADC value against a predetermined threshold value—for example one indicative of the airborne ethanol concentration being about 75 ppm. Ethanol is used in significant quantities in the manufacture of many illicit drugs, including methamphetamine, and as such it is envisaged that detection of may be used as a reliable indicator of the early stages of a clandestine drug laboratory operating in the premises in which the device is installed—or at least activity within the premises requiring investigation by the property owner or manager. It is envisaged that this threshold may avoid triggering of an alert in common household activity such as cleaning using rubbing alcohol or drinking alcohol consumption.

The controller 202 determines whether the predetermined threshold is exceeded in step 310, and if not, returns to a sleep state (depowering the sensor 218 and resetting the clock until the next reading is required). If the predetermined threshold is exceeded in step 310, an alert message is issued in step 312 to the user device 104 associated with that particular detection device 200.

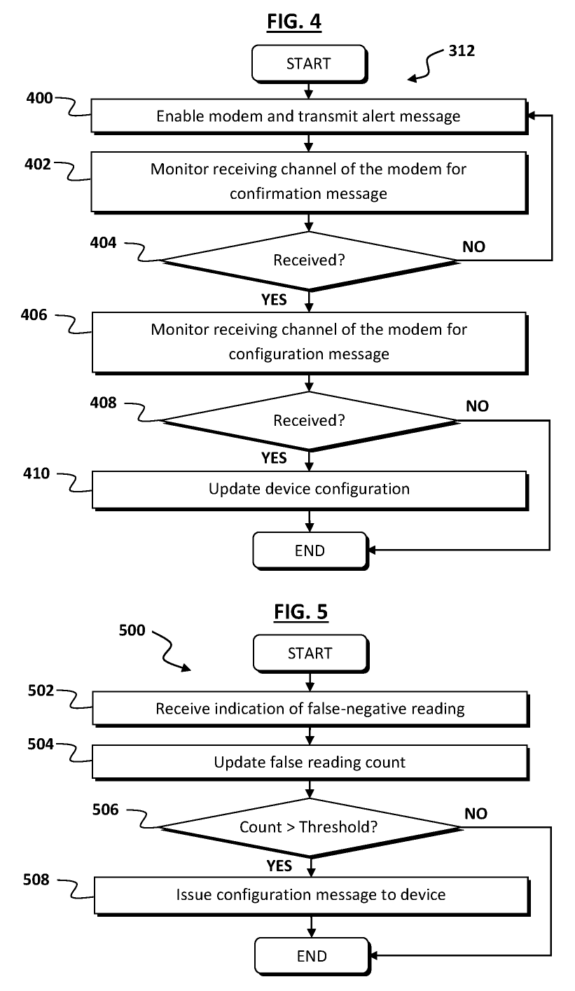

FIG. 4 illustrates an exemplary sub-process in the sending of the alert message in step 312. In step 400, on determining that the predetermined threshold has been exceeded in step 310, the controller 202 enables the modem 228 to send the alert message. In an exemplary embodiment, the controller 202 may be configured to monitor a receiving channel of the modem 228 for a predetermined period of time following issuance of the alert message. For example, in step 402 the controller 202 may monitor for a confirmation message from the user device 104 or the command centre device 108, and on determining in step 404 that a confirmation message is not received, return to step 400 and reissue the alert message.

In an exemplary embodiment, in step 406 the controller 202 may also monitor for a configuration message to adjust a configuration of the device 100, for example the predetermined threshold. On determining that a configuration message has been received in step 408, the controller 202 updates the device configuration in accordance with the configuration message in step 410.

It should be appreciated that in exemplary embodiments the step of sending of the alert message may include one, both, or neither of the steps of monitoring for the confirmation message and the configuration message. Further, it is contemplated that in exemplary embodiments a single message may include confirmation data and configuration data, and it should be appreciated that reference to the receiving of distinct messages is not intended to be limiting to all exemplary embodiments.

Once the controller 202 has determined that the alert message has been transmitted (and in exemplary embodiments has monitored for received messages), disables the modem 228 in order to preserve battery life.

FIG. 5 illustrates a method 500 of issuing a configuration message. In step 502, an indication of a false-negative reading is received—for example at an application of the user device 104, or the command centre device 108. For example, a false-negative reading may be registered by a user responding to an alert message and discovering no cause for issuance of the alert. In step 504 a count of false-negative readings is recorded, and in step 506 the count is compared with a predetermined threshold. If the predetermined threshold is not exceeded, no further action is taken.

If the predetermined threshold is exceeded, in step 508 a configuration message is issued to the device 100, for example increasing the predetermined threshold value for the sensor ADC value. At this point, the false-negative count may be reset, or reduced.

By detecting airborne ethanol in the concentrations described, it is envisaged that conditions associated with the early stages of illicit drug production, more particularly methamphetamine, may be determined before a property becomes contaminated—or at least potentially reduce the extent of contamination. The various exemplary features of the detector device described herein may assist with achieving a small physical footprint enabling the device to be installed in discreet and difficult to detect (or access) locations. Further, the various exemplary features of the detector device described herein may assist with achieving a relatively low power consumption while maintaining this small footprint.

No admission is made that any reference disclosed herein constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the field of endeavour, in New Zealand or in any other country.

Throughout this specification, the word “comprise” or “include”, or variations thereof such as “comprises”, “includes”, “comprising” or “including” will be understood to imply the inclusion of a stated element, integer or step, or group of elements integers or steps, but not the exclusion of any other element, integer or step, or group of elements, integers or steps.

Embodiments described herein may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the scope of the disclosure and without diminishing its attendant advantages. It is therefore intended that such changes and modifications be included within the present invention.

1. A device for the detection of conditions indicative of illicit drug production, including:
an airborne ethanol sensor circuit configured to output a signal indicative of airborne ethanol concentration;
a processor coupled to the airborne ethanol sensor circuit, configured to:
receive the signal indicative of airborne ethanol concentration;
determine whether a value of the signal exceeds a predetermined threshold;
a communication circuit coupled to the processor, configured to send a message to at least one remote address on determination of the value of the signal exceeding the predetermined threshold.

2. The device of claim 1, wherein the airborne ethanol sensor circuit includes an ethanol gas sensor.

3. The device of claim 2, wherein the ethanol gas sensor is a metal oxide type ethanol gas sensor.

4. The device of claim 3, wherein the metal oxide type gas ethanol gas sensor is a tin oxide (SnO₂) sensor.

5. The device of claim 2, wherein the airborne ethanol sensor circuit is coupled to a sensor driving circuit configured to deliver a pulse power up voltage to the ethanol gas sensor prior to the value of the signal indicative of airborne ethanol concentration being compared with the predetermined threshold.

6. The device of claim 2, wherein the airborne ethanol sensor circuit includes a high ohmic burden on an output of the gas ethanol sensor.

7. The device of claim 1, wherein the predetermined threshold for comparison with the value of the signal is indicative of the airborne ethanol (C₂H₅OH) concentration being above about 60 ppm.

8. The device of claim 7, wherein the predetermined threshold is indicative of the airborne ethanol (C₂H₅OH) concentration being above about 75 ppm.

9. The device of claim 7, wherein the predetermined threshold is indicative of the airborne ethanol (C₂H₅OH) concentration being above about 100 ppm.

10. The device of claim 1, wherein the processor is configured to periodically determine whether the value of the signal exceeds the predetermined threshold.

11. The device of claim 1, wherein the message sent by the communication circuit may uniquely identifies the device from which the message was sent.

12. The device of claim 1, wherein the communication circuit includes a wireless modem configured to communicate over a cellular network.

13. The device of claim 12, wherein the processor is configured to disable the modem while the value of the signal does not exceed the predetermined threshold.

14. The device of claim 12, wherein the processor is configured to enable the modem and send status messages at predetermined instances when the value of the signal does not exceed the predetermined threshold.

15. The device of claim 12, wherein the processor is configured to enable the modem and listen for command messages at predetermined instances.

16. The device of claim 15, wherein the processor is configured to listen for command messages for a predetermined period of time following sending the message.

16. The device of claim 15, wherein the processor is configured to listen for command messages for a predetermined period of time following sending the message.

17. The device of claim 1, including a self-contained power source.

18. A method for the detection of conditions indicative of illicit drug production, including:
driving an airborne ethanol sensor circuit to output a signal indicative of airborne ethanol concentration;
receiving the signal indicative of airborne ethanol concentration at a processor;
determining whether a value of the signal exceeds a predetermined threshold; and
sending a message to at least one remote address on determination of the value of the signal exceeding the predetermined threshold.

19. The method of claim 18, wherein driving the airborne ethanol sensor circuit includes delivering a pulse power up voltage to an ethanol gas sensor of the airborne ethanol sensor circuit prior to the value of the signal indicative of airborne ethanol concentration being compared with the predetermined threshold.

20. The method of claim 18, wherein the predetermined threshold for comparison with the value of the signal is indicative of the airborne ethanol (C₂H₅OH) concentration being above about 60 ppm.

21-26. (canceled)
